(19) United States
(12) Patent Application Publication
(10) Pub. No.: US 2011/0028533 A1
(43) Pub. Date: Feb. 3, 2011

(54) NOVEL FER -LIKE PROTEIN, PHARMACEUTICAL COMPOSITIONS CONTAINING IT AND METHOD FOR ITS USE

(75) Inventors: Uri Nir, Moshav Gamzo (IL); Adar Makovsky, Yahood (IL); Sally Shpungin, Ramat-Gan (IL)

Correspondence Address: THE NATH LAW GROUP, 112 South West Street, Alexandria, VA, 22314, US

(73) Assignee: URIFER LTD., Raanana (IL)

(21) Appl. No.: 12/866,403

(22) PCT Filed: Feb. 5, 2009
(86) PCT No.: PCT/IL2009/000136

§ 371 Date: Aug. 5, 2010

Related U.S. Application Data
(60) Provisional application No. 61/006,897, filed on Feb. 5, 2008.
(60) Provisional application No. 61/129,112, filed on Jun. 5, 2008.

(51) Int. Cl. A61K 31/711; C07H 21/04; G01N 33/53; G01N 33/574; C07K 14/00; A61P 35/00

(52) U.S. Cl. 514/44 A; 536/24.5; 435/7.21; 435/7.23; 530/350; 530/324; 536/23.1

(57) ABSTRACT

Provided is a novel Fer-like protein, referred to as “FerC” (Fer colorectal cancer). FerC is a 47kDa protein having a unique N-terminal sequence and was found to be present in six colon cancer cell-lines and in five hepatocarcinoma (liver cancer) cell-lines, but not in CCD33 normal colon epithelial cells or normal human and mouse fibroblasts. Depletion of FerC impairs cell-cycle progression and induces apoptotic death in treated colon cancer (CC) cells. Also provided are nucleotide sequences that are antisense to at least a portion of the N-terminal sequence, short interfering nucleotides including such an antisense sequence, as well as pharmaceutical compositions containing an antisense sequence. The present pharmaceutical composition may be used in the treatment of cancer, in particular, colorectal cancer and liver cancer.

### FIELD OF THE INVENTION

This invention relates to pharmaceutical compositions, and more specifically to such compositions for the treatment of cancer.

### BACKGROUND OF THE INVENTION

The following prior art publications are considered as being relevant for an understanding of the invention.
Allard P, Zoubeidi A, Nguyen L T, Tessier S, Tanguay S, Chevrette M, Aprikian A and Chevalier S. (2000). Mol. Cell. Endocrinol., 159, 63-77.
Ben-Dor I, Bern O, Tennenbaum T and Nir U. (1999). Cell Growth Differ., 10, 113-129.
Craig A W, Zirngibl R, Williams K, Cole L A and Greer P A. (2001). Mol. Cell. Biol., 21, 603-613.
Delfino F J, Stevenson H and Smithgall TE. (2006). J. Biol. Chem., 281, 8829-8835.
Fischman K, Edman J C, Shackleford G M, Turner J A, Rutter W J and Nir U. (1990). Mol. Cell. Biol., 10, 146-153.
Gascoigne K E and Taylor S S. (2008). Cancer Cell, 14, 111-122.
Greer P. (2002). Nat. Rev. Mol. Cell Biol., 3, 278-289.
Halachmy S, Bern O, Schreiber L, Carmel M, Sharabi Y, Shoham J and Nir U. (1997). Oncogene, 14, 2871-2880.
Hao Q-L, Heisterkamp N and Groffen J. (1989). Mol. Cell. Biol., 9, 1587-1593.
Hazan B, Bern O, Carmel M, Lejbkowicz F, Goldstein R S and Nir U. (1993). Cell Growth Differ., 4, 443-449.
Kaufmann S H, Desnoyers S, Ottaviano Y, Davidson N E and Poirier GG. (1993). Cancer Research, □□, 3976-3985.
Keshet E, Itin A, Fischman K and Nir U. (1990). Mol. Cell. Biol., 10, 5021-5025.
Letwin K, Yee S-P and Pawson T. (1988). Oncogene, 3, 621-627.
Orlovsky K, Ben-Dor I, Priel-Halachmi S, Malovany H and Nir U. (2000). Biochemistry, 39, 11084-11091.
Pasder O, Shpungin S, Salem Y, Makovsky A, Vilchick S, Michaeli S, Malovani H and Nir U. (2006). Oncogene, 25, 4194-4206.
Polyak K, Waldman T, He T C, Kinzler K W and Vogelstein B. (1996). Genes Dev., 10, 1945-1952.
Priel-Halachmi S, Ben-Dor I, Shpungin S, Tennenbaum T, Molavani H, Bachrach M,
Salzberg S and Nir U. (2000). J. Biol. Chem., 275, 28902-28910.
Salem Y, Shpungin S, Pasder O, Pomp O, Taler M, Malovani H and Nir U. (2005). Cell Signal., 17, 341-353.
Sangrar W, Gao Y, Scott M, Truesdell P and Greer P A. (2007). Mol. Cell Biol., 27, 6140-6152.
Sangrar W, Zirgnibl R A, Gao Y, Muller W J, Jia Z and Greer P A. (2005). Cancer Res., 65, 3518-3522.
van Engeland M, Ramaekers F C, Schutte B and Reutelingsperger C P. (1996). Cytometry, □□, 131-139.

Fer is an intracellular tyrosine kinase which was found to reside in both the cytoplasm and nucleus of mammalian cells (Ben-Dor et al., 1999; Hao et al., 1989; Letwin et al., 1988). Together with c-Fes, Fer represents a distinct subfamily of intracellular tyrosine kinases that share a unique structure. Both kinases bear an extended N-terminal tail which contains an Fps/Fes/Fer/CIP4 homology domain (FCH), followed by three coiled-coil-forming regions (Greer, 2002). A truncated variant of Fer, termed FerT, uniquely accumulates in meiotic spermatogenic cells (Fischman et al., 1990; Hazan et al., 1993; Keshet et al., 1990). Fer and FerT share common kinase and SH2 domains but they differ in their N-terminal tail where the 412 amino-acid-long tail in Fer is replaced with a unique 43 amino-acid-long tail in FerT (Priel-Halachmi et al., 2000).

Although present in a wide variety of tissues and cells, mice devoid of active Fer develop normally, and the proliferation of fibroblasts derived from these mice is not impaired in vitro (Craig et al., 2001). However, Fer has been implicated in the response of cells to stress cues. Fer was shown to rescue cells from ionic radiation (Halachmy et al., 1997), to support their growth under oxygen deprivation (Salem et al., 2005), and to mediate the migratory response of fibroblasts to reactive oxygen species (Sangrar et al., 2007). The role of Fer in supporting cell growth under abnormal conditions is further manifested in malignant cells. Several lines of evidence suggest a supportive role of Fer in the progression and growth of malignant tumors. Fer was detected in all human malignant cell lines analyzed (Hao et al., 1989; Orlovsky et al., 2000) and its levels in malignant prostate tumors are significantly higher than those detected in benign tumors (Allard et al., 2000). Furthermore, down-regulation of Fer impairs the proliferation of prostate and breast carcinoma cells (Pasder et al., 2006) and abolishes the ability of prostate carcinoma PC3 cells to form colonies in soft agar (Allard et al., 2000). This pro-oncogenic activity of Fer differs from the tumor suppressive activity of C-Fes in colon cancer (CC) cells (Delfino et al., 2006; Sangrar et al., 2005).

### SUMMARY OF THE INVENTION

The present invention is based on the novel and unexpected finding of a novel Fer-like protein, referred to herein as “FerC” (Fer colorectal cancer). FerC is a 47 kDa protein having the sequence SEQ ID NO: 12 that contains the SH2 and kinase domains of Fer, but has a novel N-terminal sequence (SEQ ID NO: 14). FerC was found to be present in six colon cancer cell-lines analyzed, and in the hepatocarcinoma (liver cancer) cell-lines: SK-Hep-1, Hep-G2, Huh-6, Huh-7 and Hep-3B. FerC was not detected in HT29 adenocarcinoma cells, CCD33 normal colon epithelial cells or normal human and mouse fibroblasts.

Depletion of FerC impairs cell-cycle progression and induces apoptotic death in treated colon cancer (CC) cells, an effect that was exacerbated by the simultaneous knockdown of both FerC and Fer. Cell viability in HCT116 cell cultures following depletion of one or both of the Fer and FerC proteins indicated that simultaneous knockdown of Fer and FerC decreases by two fold the percentage of viable cells in the treated cultures.

Knockdown of FerC interfered with cell-cycle progression in HCT116 and RKO cells, but also induced apoptotic cell death, manifested by increased accumulation of the sub-G1 population in the treated cells, an effect that was enhanced upon the simultaneous knockdown of Fer and FerC.

Thus, in one of its aspects, the invention provides a polypeptide having the amino acid sequence SEQ ID NO 12. The invention also provides a polypeptide having the amino acid sequence SEQ ID NO 14. The polypeptides of the invention may be used, for example, to generate antibodies reacting with either the peptide of SEQ ID NO 12 or with the peptide of SEQ ID NO 14. Such antibodies can be used to detect the presence of either one of the peptides of SEQ ID NO 12 or SEQ ID NO 14, for example in a western blot analysis. The invention further provides a nucleotide sequence encoding the polypeptide SEQ ID NO 12 and a nucleotide sequence encoding the polypeptide SEQ ID NO 14. Such nucleotides can be used, for example, to produce the peptides of SEQ ID NO 12 or SEQ ID NO 14. The nucleotide sequences of the invention may be deoxyribonucleic nucleic acids, ribonucleic acids, or a mixture of deoxy- and ribo-nucleic acids, as well as synthetic nucleic acids. In a preferred embodiment, the nucleotide sequence encoding the polypeptide SEQ ID NO 12 is the nucleotide sequence of SEQ ID NO. 11. In a preferred embodiment, the nucleotide sequence encoding the polypeptide SEQ ID NO 14 is the nucleotide sequence of SEQ ID NO. 13.

In another of its aspects, the invention provides a nucleotide sequence comprising a sequence that is antisense to at least a portion of the nucleotide sequence SEQ ID NO: 13 and capable of specifically binding to SEQ ID NO. 13. The antisense sequence preferably has a length of at least 10 nucleotides, and mot preferably, a length of at least 19 nucleotides. In a preferred embodiment, the antisense nucleotide sequence is SEQ ID NO: 9. The invention also provides a short interfering nucleotide sequence comprising such an antisense nucleotide sequence. The invention further provides a pharmaceutical composition comprising a nucleotide sequence a sequence that is antisense to at least a portion of the nucleotide sequence SEQ ID NO: 13 and capable of specifically binding to SEQ ID NO. 13, and a physiologically acceptable carrier. The pharmaceutical composition of the invention may contain a short interfering nucleotide sequence comprising a nucleotide sequence that is antisense to at least a portion of the nucleotide sequence SEQ ID NO: 13 and capable of specifically binding to SEQ ID NO. 13. The pharmaceutical composition may be used in the treatment of various cancers, in particular, colorectal cancer and liver cancer. The pharmaceutical composition of the invention may further comprise a nucleotide sequence comprising a sequence that is antisense to at least a portion of the fer gene. Preferably, the sequence that is antisense to at least a portion of the fer gene is SEQ ID NO 8.

In yet another of its aspect, the invention provides a method for identifying a cancerous state in a cell comprising detecting FerC in the cell. The method preferably comprises:
(a) extracting proteins from the cell;
(b) detecting Fer94 in the protein extract using a first antibody reactive with Fer94 and FerC;
(c) detecting Fer94 in the protein extract using a second antibody reactive with Fer94 and not reactive with FerC;
   the presence of a protein in the extract reactive to the first antibody and not reactive to the second antibody being indicative of the presence of FerC in the cell.

The second antibody preferably binds to an epitope located at the N-terminus of Fer94. The invention also provides a kit for identifying a cancerous state in a cell comprising:
(a) a first antibody reactive with Fer94 and FerC;
(b) a second antibody reactive with Fer94 and not reactive with FerC.

The invention further provides a method of treating cancer comprising administering to an individual a pharmaceutical composition of the invention. The cancer may be, for example, colorectal cancer, or liver cancer.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1A shows western blot analysis of whole cell lysates of normal human colon epithelial cells-CCD33 (lane 1), and from seven other human CC-lines (lanes 2-8) after reaction of SDS-PAGE resolved proteins with anti-SH2-Fer antibodies; FIG. 1B shows western blot analysis of lysates from HT29 and from HCT116 of SDS-PAGE resolved proteins reacted with antibodies directed toward the unique N-terminal tail of Fer (1-189 aa) (left panel), or toward the C-terminal end of Fer (right panel); FIG. 1C shows. detection with anti-SH2-Fer antibodies of: HCT116 whole cell lysates (1); normal human colon tissue (2,3); stage II primary colorectal adenocarcinoma (4,5); CC metastases to the liver (6); CC metastases to the lung (7); CC metastases to the ovary (8); and stage I adenocarcinoma (9);

FIG. 2 A shows schematically the structure of the FerC protein, and FIG. 2 B shows an RT-PCR product of 1420 by that demonstrates the preferential accumulation of ferC mRNA in HCT116 cells;

FIG. 3 A shows knock down of Fer and FIG. 3 B shows FerC, in HCT116 and RKO CC to cells using 50 nM specific and selective siRNAs; and FIG. 3 C shows transfection of HCT116 with different siRNAs and evaluation by the XTT cell viability kit (Biol. Ind., Israel);

FIG. 4 A shows the distribution of HCT116, and FIG. 4 B shows the distribution of RKO cells, in the various cell-cycle fractions, upon treatment with siRNAs directed toward the fer and ferC RNAs; FIG. 4 C shows incorporation of BrdU in CC cells following down-regulation of Fer or FerC; FIG. 4 D shows staining of HCT116 Cells (2×10⁵) with Annexin V-FITC and propidium iodide after transfection with the siRNAs; FIG. 4 E shows lysates of HCT116 cells transfected with different siRNAs; and FIG. 4 F shows resolution of whole cell protein lysates from Fer and FerC depleted HCT116 cells, which were resolved by SDS-PAGE and reacted with anti-p53, anti-p21, anti-cdc2 anti-actin antibodies, in a western blot analysis.

### EXPERIMENTAL RESULTS

Seven human CC cell-lines derived from various tumor stages, and from normal, human colonic epithelial cells (CCD33) were obtained from the ATCC and grown according to the ATCC instructions. Whole cell lysates were prepared from the cells and resolved by 10% SDS-PAGE (30 μg protein from each sample) and were then reacted with specific, anti-SH2-Fer antibodies (Priel-Halachmi et al., 2000) using western blot analysis.

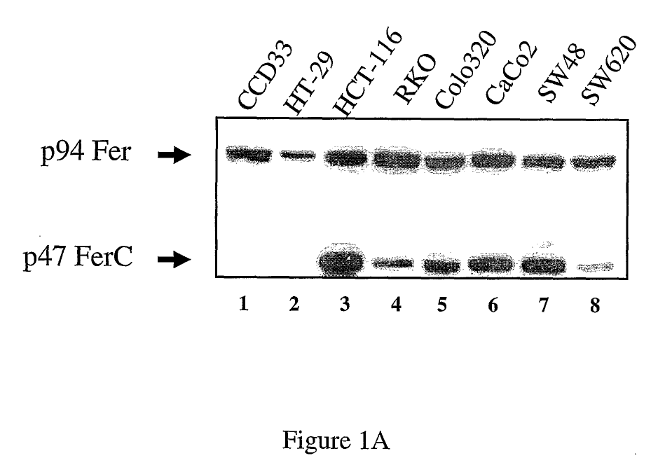

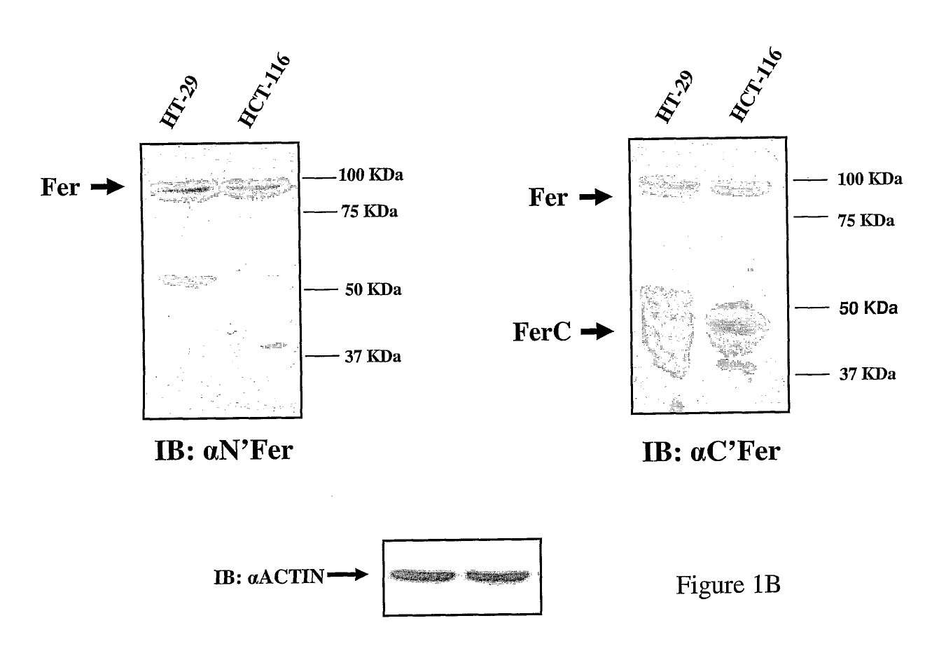

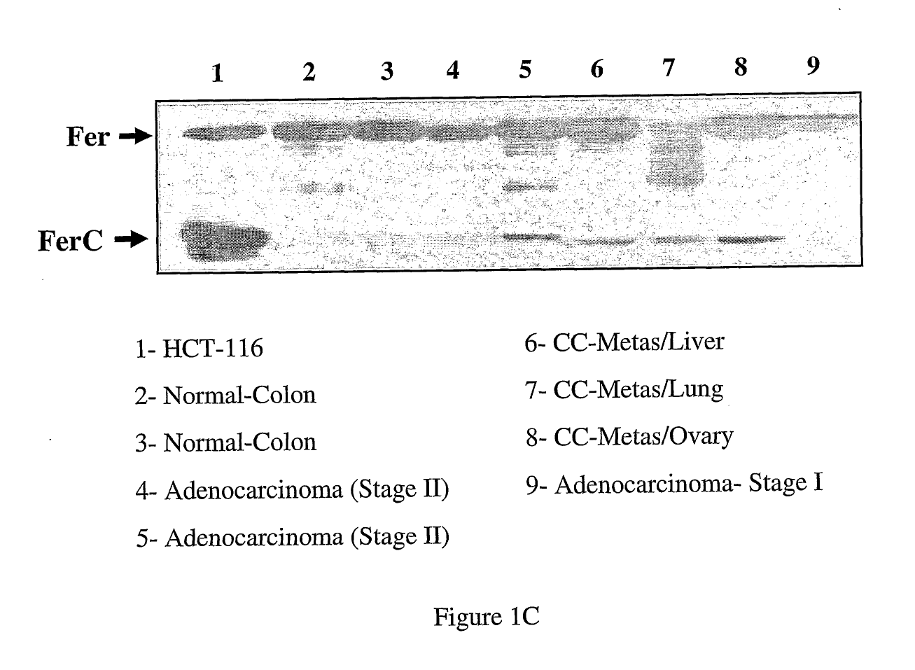

FIG. 1 shows the expression profile of Fer in CC cells. In FIG. 1A, shows the western blot analysis of the normal human colon epithelial cells-CCD33 (lane 1), and from the seven human CC-lines (lanes 2-8), as indicated in FIG. 1A. In six of the CC cell-lines analyzed, an additional protein of about 47 kDa molecular weight reacted with the specific, anti-SH2-Fer antibodies. The 47 kDa was not detected in either the HT29 adenocarcinoma cells, or in the CCD33 normal colon epithelial cells. This 47 kDa protein was also absent from normal human and mouse fibroblasts (data not shown).

To gain an indication whether the 47 kDa protein represents a truncated variant of Fer, lysates from the CC cells were reacted with anti-Fer antibodies directed toward either the N-terminal tail or the C-terminal end of the protein. In FIG. 1B, lysates from HT29 and from HCT116 cells were reacted with antibodies directed toward the unique N-terminal tail of Fer (1-189 aa) (left panel), or antibodies directed toward the C-terminal end of Fer (right panel). α-actin served as protein quantity control (anti-actin, Sigma). While anti-C terminus antibodies reacted with the 47 kDa protein, the anti-N terminal antibodies reacted only with the 94 kDa full length Fer. These results suggested the accumulation in CC cells of a truncated variant of Fer, which lacks the N-terminal tail of the somatic kinase. This Fer variant is referred to herein as “FerC” (Fer colorectal cancer).

To examine whether the presence of FerC in CC cell-lines reflects the accumulation of this protein in a tumor specific manner, whole cell lysates from normal colon, primary CC tumors and from lung, liver and ovarian metastases of CC tumors were resolved by SDS-PAGE and were then reacted with anti-SH2-Fer antibodies. Lysates from primary colonic tissues or tumors were purchased from Origene Inc. (origene.com-HCRT1). FIG. 1C shows detection in lysates with anti-SH2-Fer antibodies of: HCT116 whole cell lysates (lane 1); normal human colon tissue (lanes 2,3); stage II primary colorectal adenocarcinoma (lanes 4,5); CC metastases to the liver (lane 6); CC metastases to the lung (lane 7); CC metastases to the ovary (lane 8); stage I adenocarcinoma (lane 9). The anti-SH2-Fer antibodies detected the p94Fer in all seven CC cell-lines as well as in the normal CCD33 cells. FerC was not detected in normal colon tissues nor in a stage I colon adenocarcinoma lysate. However, FerC was detected in primary stage II CC tumors. Furthermore, FerC was also present in CC metastases to the liver, lungs and ovaries.

In order to characterize and identify the Fer-related 47kD protein, RNAs containing Fer-related sequences were cloned from HCT116 and HT29 cells. RACE was performed by using a 5′/3′ RACE kit (2nd generation, Roche) following the manufacturer's instructions. The specific primers used were:

(SEQ. ID NO. 1)
Sp1: 5′-TCCCTTGCCCAGTAATTCTCCCAATATGAC-3′
(SEQ. ID NO. 2)
Sp2: 5′-AACCCAGTGCCCTCGAATCGATAC-3′
(SEQ. ID NO. 3)
Sp3: 5′-GGACATATTCACCAGGTTTCCCATGACTCTC-3′.

The RACE product was analyzed on a 1% ethidium bromide stained agarose gel, and was cloned into the pGEM-T easy vector system (Promega). Tthe nucleotide sequence of the ferC cDNA is shown in SEQ ID NO: 11, and the longest open reading frame (ORF) translation product is shown in SEQ ID NO: 12. The unique N-terminal sequence is shown in SEQ ID NO: 13. The longest ORF of the ferC cDNA from HCT116 cells was found to encode a truncated Fer protein. FerC is 453 aa in length and has a calculated MW of 51 kDa, though it migrates in PAGE as a 47 kDa protein. FerC contains intact SH2 and kinase domains of Fer, which are linked to a unique 43 amino acid long N-terminal tail.

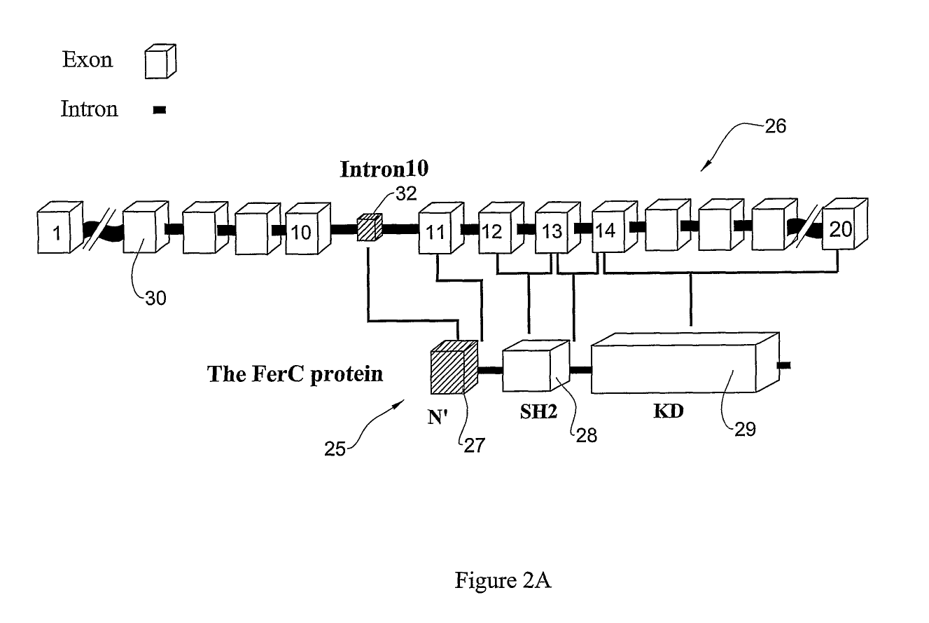

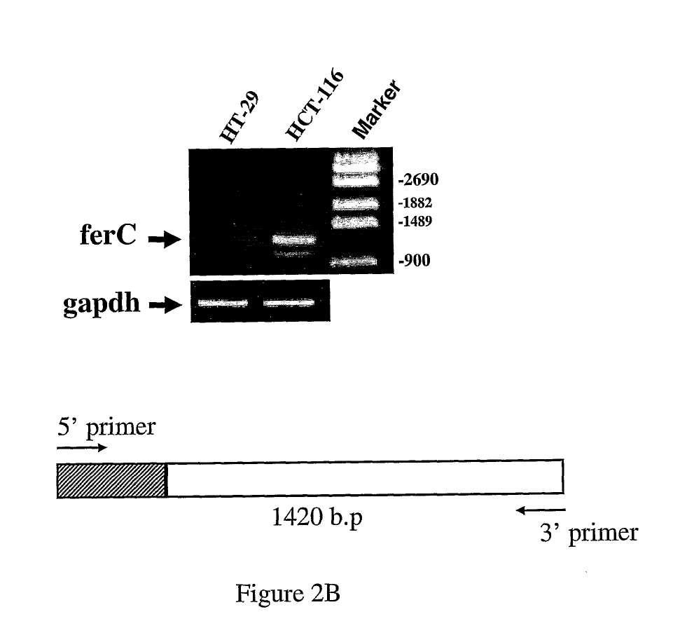

FIG. 2A shows schematically the structure of the FerC 25 protein in comparison to p94 Fer 26. The unique N-terminal tail 27, the SH2 domain 28 and the kinase domain (KD) 29 are depicted. Exons of the fer locus encoding the Fer and FerC proteins are indicated by the cubes 30, which are numbered according to the exon number, and correspond to the encoded domains of FerC. Alignment of the ferC cDNA sequence with the human fer locus indicated that the unique N-terminal tail of FerC is encoded by intron 10 of the fer gene. The first nucleotide of the translation initiation codon of the ferC mRNA is transcribed from nucleotide 26,813 in intron 32 of the human fer locus. This intron is comprised of 48,281 nucleotides [ensemble human gene view]. The remainder of the FerC protein is encoded by exons 11-20 of the fer gene.

To confirm the link between the cloned ferC RNA and the 47 kDa FerC protein, a semi-quantitative RT-PCR analysis was performed on RNA prepared from HCT116 and from HT29 cells. This was carried out using a 3′ primer which is common to fer and ferC, and a unique primer corresponding to the 5′ end of the ferC cDNA. RNA was extracted from HT29 and from HCT116 cells using TRI Reagent (Mol. Res. Center, Aurora, Ohio) following the manufacturer's instructions. 1 μg total RNA was reverse-transcribed using the SuperScript first-strand synthesis system for RT-PCR (Invitrogen). For semi-quantitative RT-PCR analysis, the PCR product was amplified using 35 cycles with the following primers derived from the human ferC cDNA:

(SEQ. ID NO. 4)
5′-CCACATCAGAAGTCCACAGAGATCAGGAAAG-3′
(SEQ. ID NO. 5)
5′-GCCCGCGAATTCACACTCAAAAGAGAACTAC-3′

The following gapdh RNA served as an internal control:

| | |
|---|---|
| 5′-AAGGTCATCCCTGAGCTGAACG-3′ | (SEQ. ID NO. 6) |
| 5′-CAAAGGTGGAGGAGTGGGTGTC-3′. | (SEQ. ID NO. 7) |

PCR products were separated on a 1% agarose gel and stained with ethidium bromide.

FIG. 2B shows, consistent with the accumulation of FerC in HCT116 cells, the existence of an RT-PCR product of 1420 by and demonstrates the preferential accumulation of the ferC mRNA in HCT116 cells.

To gain insight into the function of the Fer proteins in CC cells, the Fer and FerC were simultaneously or individually knocked-down in the colon carcinoma cell-lines HCT116 and RKO, which express both the intact Fer and the truncated FerC.

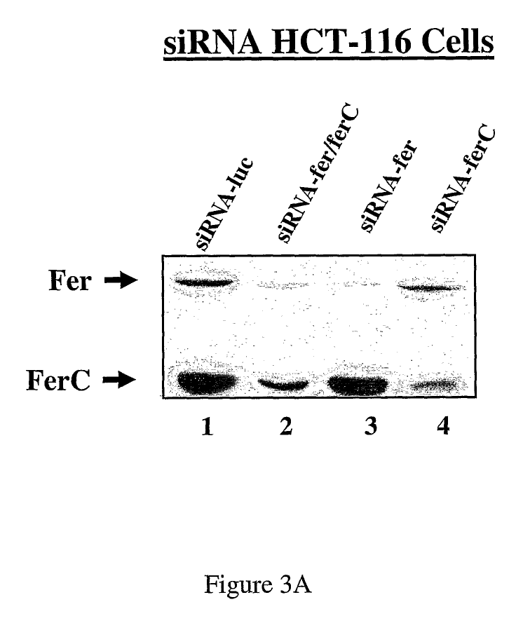

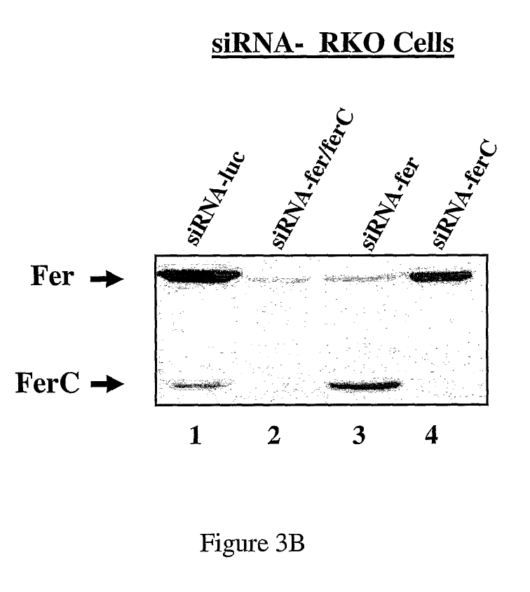

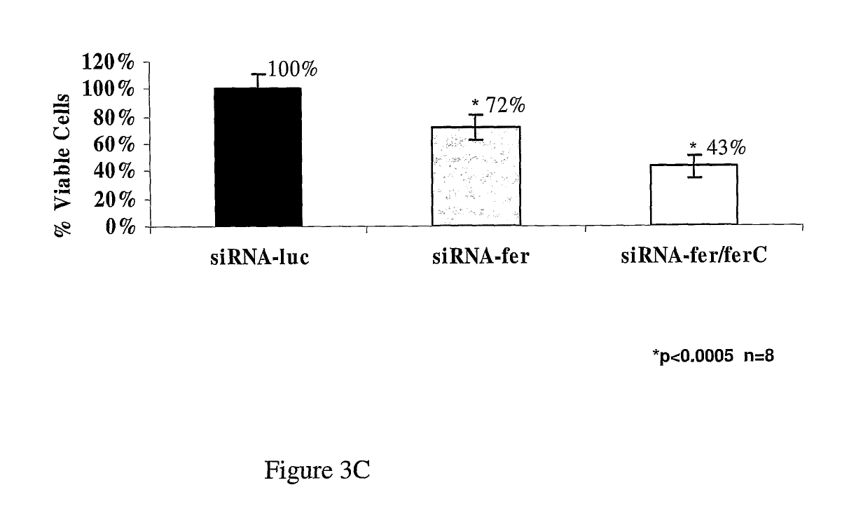

Fer or FerC was selectively knocked down in HCT116 and RKO CC cells using 50 nM specific and selective siRNAs directed toward the unique 5′ sequences of the fer (siRNA-fer:5′-ACGUAUCCAAGUCUUGGCUACUUAU-3′, SEQ. ID NO. 8) or ferC (siRNA-ferC:5′-CAGCUCUGAGCCUUCCACAUCAGAA-3′, SEQ. ID NO. 9). A fer-specific siRNA directed towards a common sequence present in the fer and ferC RNAs (siRNA-fer/ferC: 5′-GCCCUAAGUUCAGUGAACUUCAGAA-3′, SEQ. ID NO. 10) was used for simultaneous knockdown of Fer and FerC. A sequence targeting luciferase (siRNA-luc) was used as a non-relevant siRNA negative control (Dharmacon). 2×10⁵ cells were transfected with siRNAs using the Lipofectamine 2000 reagent, according to the manufacturer's instructions (Invitrogen). As shown in FIGS. 3A and 3B, lanes 3 and 4, the specific siRNAs directed toward the unique 5′ sequences of the fer and ferC mRNAs selectively knocked-down Fer or FerC, respectively.

Cell viability in HCT116 cell cultures following depletion of one or both of the Fer and FerC proteins was studied. HCT116 Cells were seeded in six well plates (2×10⁵ cells per well). 48 h after transfection with the different siRNAs, cell viability was evaluated by using the XTT cell viability kit (Biol. Ind., Israel) following the manufacturer's instructions. The absorbance at 450 nm was measured using a microplate reader (Spectra Fluor Plus-Tecan. Inc.). Statistical analysis was performed using the paired and unpaired Student's t-test, with a P value ≧0.05 being considered significant. The results are shown in FIG. 3C as mean±standard error (SE) of the mean for 8 samples. Simultaneous knockdown of Fer and FerC in HCT116 cells decreased by two fold the percentage of viable cells in the treated cultures. Similar results were obtained upon knockdown of the Fer proteins in RKO cells (data not shown).

To determine whether Fer and FerC support the proliferation or survival of CC cells, cultures treated with siRNAs directed toward the fer and ferC mRNAs were subjected to flow-cytometric analysis. HCT116 cells were transfected with siRNAs for 48 h and the percentage of cells incorporating BrdU was determined. Cell cycle analysis and BrdU incorporation assays were performed as described in Pasder et al., 2006.

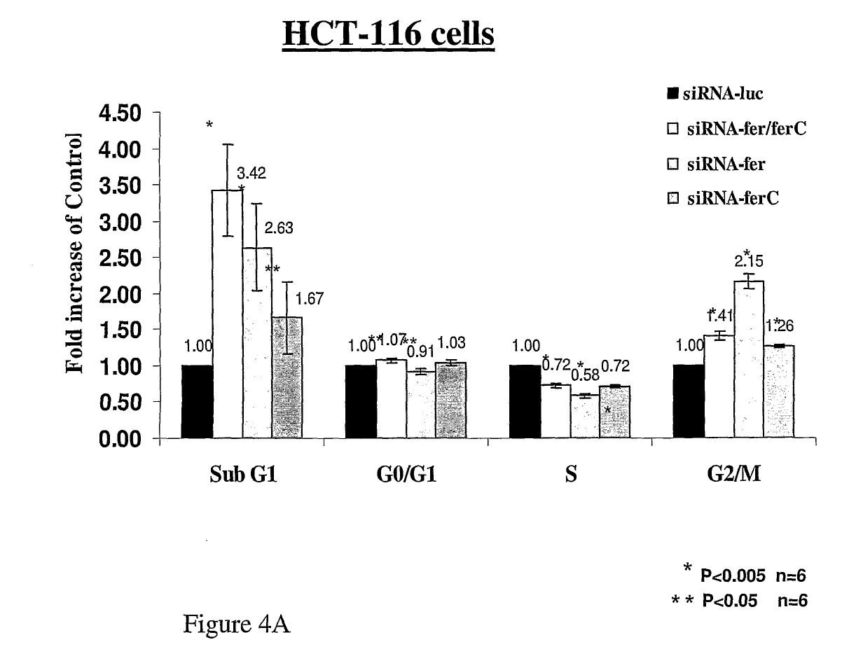

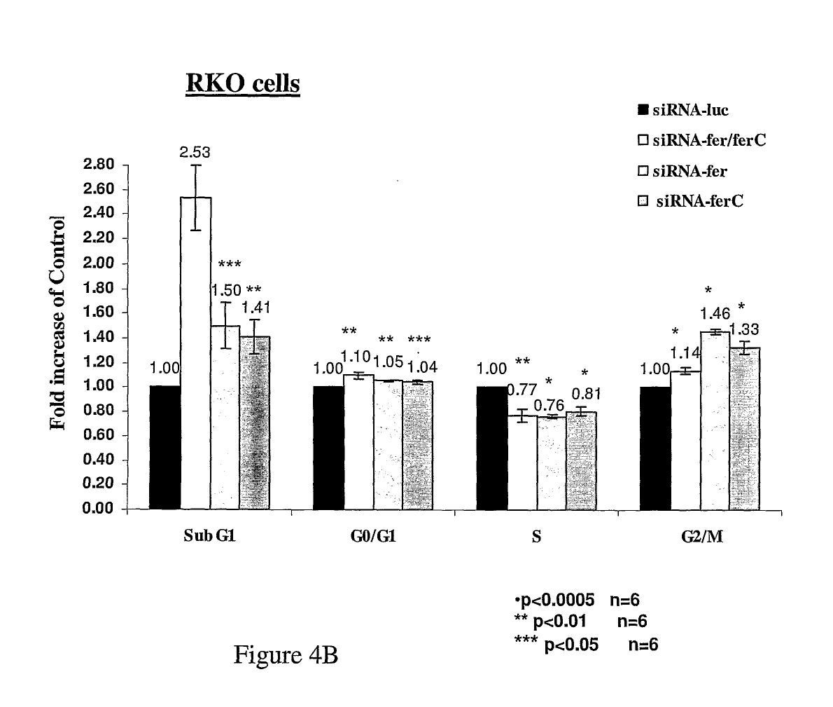

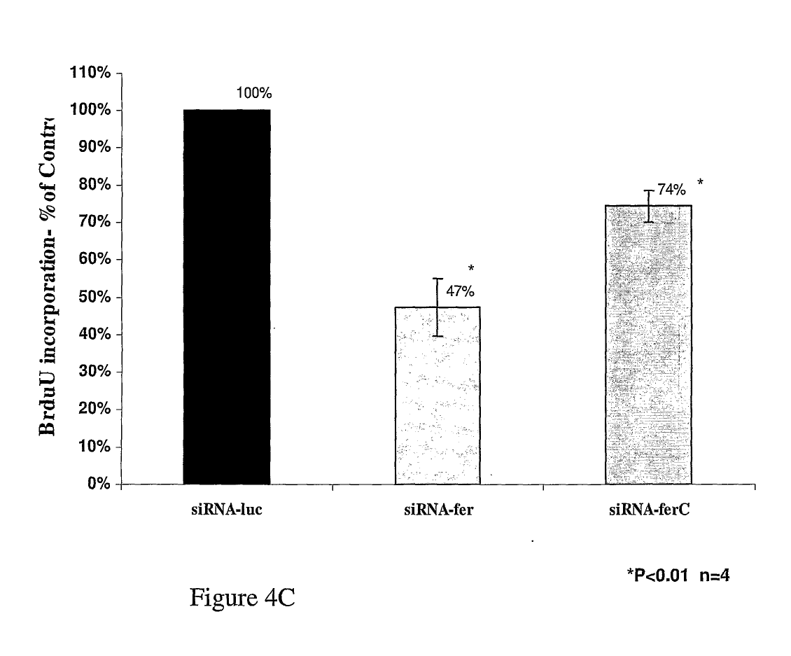

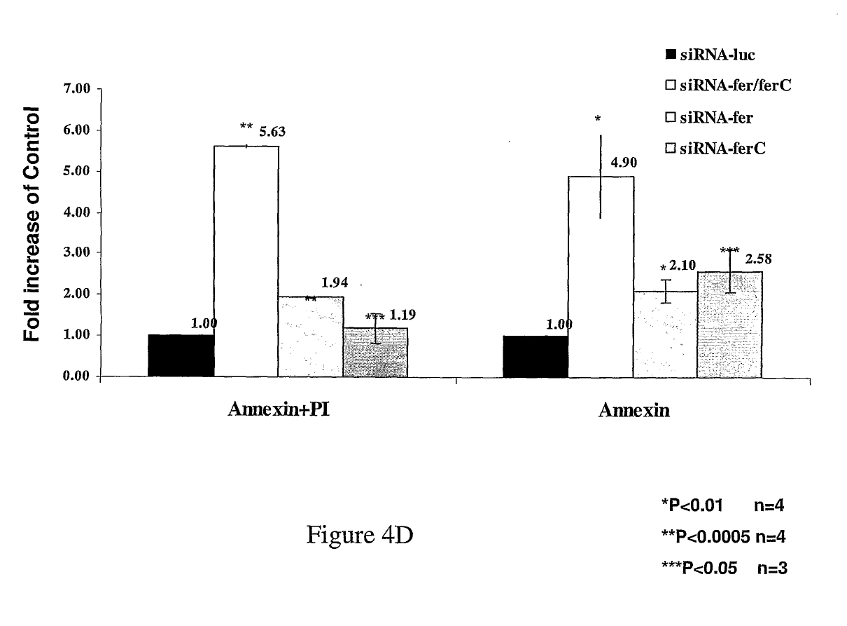

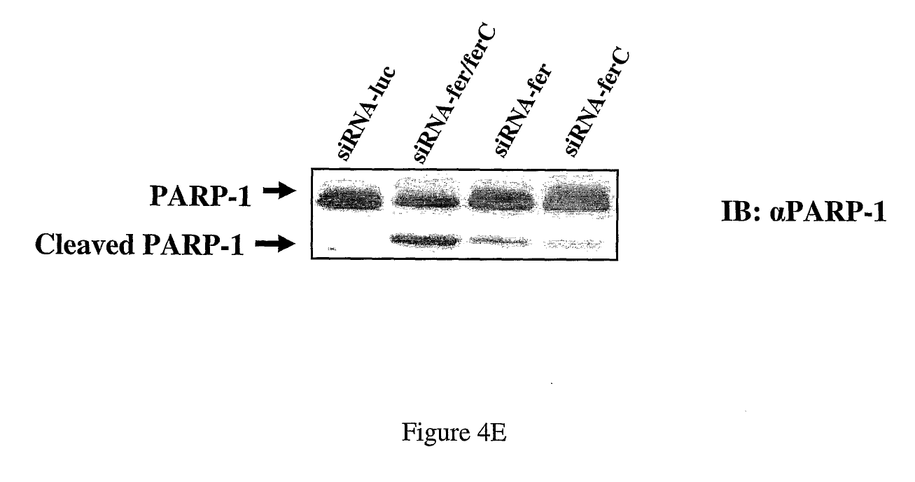

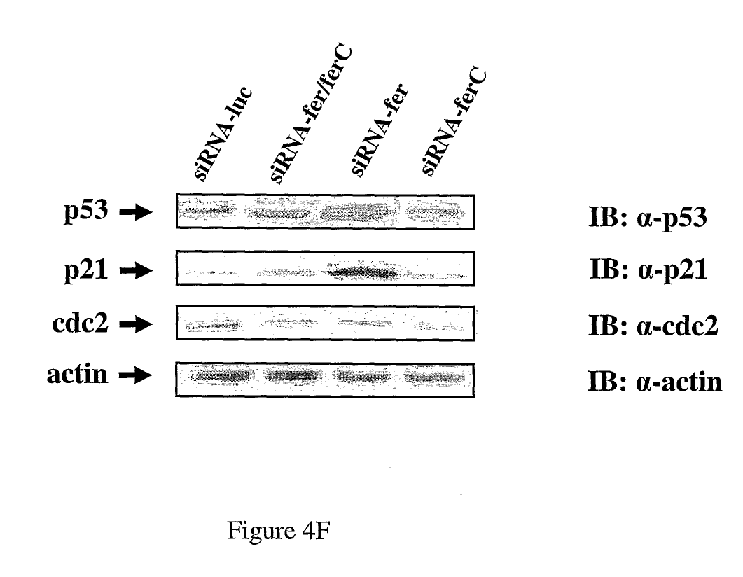

The distribution of HCT116 and RKO cells in the various cell-cycle fractions, upon treatment with siRNAs directed toward the fer and ferC RNAs is shown in FIGS. 4A and 4B. Changes in the sub-G0/G1 fraction are shown, as well. Selective knockdown of Fer or FerC decreased the percentage of cells residing in the S phase and increased the fraction of G2/M cells. This effect was more profound in Fer-depleted cells and was not enhanced upon the simultaneous knockdown of the two proteins. Notably, although the percentage of S phase cells was significantly decreased upon the knockdown of Fer or FerC, there was no significant increase seen in the G0/G1 fraction of the treated cells. This most probably is a result of the significant increase in the sub-G1 fraction of cells depleted of Fer or FerC, and could reflect a transition of G2/M arrested cells to apoptotic death rather than to the G0/G1 phase (Gascoigne and Taylor, 2008). Furthermore, the significant increase of the sub-G1 fraction upon the combined depletion of Fer and FerC could explain the lack of additive effects on the cell-cycle profile by the simultaneous knockdown of the two Fer proteins.

To substantiate the attenuation of the G1-S transition as a result of the knockdown of Fer or FerC, the percentage of BrdU incorporating cells was determined following Fer or FerC depletion. The results, shown in FIG. 4C, demonstrated a significant reduction in the level of BrdU incorporation upon the knockdown of Fer or FerC. Thus, the two Fer variants sustain the progression of both the G1-S and G2-M transition points in CC cells.

As noted above, knockdown of Fer or FerC not only interfered with cell-cycle progression in HCT116 and RKO cells, but also induced apoptotic cell death. This effect was manifested by increased accumulation of the sub-G1 population in the treated cells, an effect that was enhanced upon the simultaneous knockdown of Fer and FerC (FIGS. 4A and 4B). The profound induction of apoptotic death upon the simultaneous knock-down of Fer and FerC in CC cells was further demonstrated by staining the treated cells with Annexin V, which stains membranes of apoptotic cells (van Engeland et al., 1996) and by the identification of cleaved Poly(ADP-ribose) polymerase-1 (PARP-1) (Kaufmann et al., 1993) in the Fer and FerC depleted cells. For Annexin V staining, HCT116 Cells were plated in six well plates (2×10⁵ cells per well), and 48 h after transfection with siRNA were stained with Annexin V-FITC and propidium iodide using the MEBCYTO-apoptosis kit (MBL) following the manufacturer's instructions. The total cellular DNA content and bound Annexin V-FITC were determined using a Becton Dickinson flow cytometer (FACS Calibur), and all data were analyzed using the Cell Quest Pro software. The fractions of cells stained with both Annexin and propidium iodide (PI) is shown in the left histogram of FIG. 4D, and the fractions of cells stained with Annexin but not with PI is shown in the right histogram. Values represent means±SE of Annexin-V staining in four independent experiments.

For the identification of cleaved PARP-1, lysates were resolved by SDS-PAGE and were then reacted with anti-PARP-1 antibody (Santa-Cruz) in a western-blot analysis.

The results are shown in FIG. 4E, which represents one out of three independent experiments which gave similar results. Migration distances of the intact and cleaved PARP-1 are shown.

To unveil whether the apoptotic death invoked in the Fer and FerC depleted cells is linked to the induction of the transcription factor p53, the level of p53 was compared in the various treated cells. Fer and FerC were separately or simultaneously knocked-down in HCT116 cells with the different siRNAs. Lysates were prepared and resolved in SDS-PAGE and reacted with anti-p53, anti-p21, anti-cdc2 anti-actin antibodies (Santa-Cruz), in a western blot analysis. As shown in FIG. 4F, knock-down of Fer significantly up-regulated the level of p53 and concomitantly also the level of its down-stream effector p21, which acts as a negative regulator of proliferation and apoptosis in HCT116 cells (Polyak et al., 1996). In parallel, the level of another target of p53, the G2/M transition regulator cdc2, was decreased. However, simultaneous depletion of Fer and FerC interfered with the accumulation of p53 and its effects on the down-stream targets p21 and cdc2, thus allowing a shift from cell-cycle arrest to an increase in apoptotic death. FIG. 4E represents one out of three independent experiments which gave similar results.

1-23. (canceled)

24. A nucleotide sequence comprising a sequence that is antisense to at least a portion of the nucleotide sequence SEQ ID NO: 13 and capable of specifically binding to SEQ ID NO. 13.

25. The nucleotide sequence according to claim 24, having a length of at least 10 nucleotides.

26. The nucleotide sequence according to claim 24, having a length of at least 19 nucleotides.

27. The nucleotide sequence according to claim 24, being SEQ ID NO: 9.

28. A short interfering nucleotide sequence comprising the nucleotide sequence according to claim 24.

29. A pharmaceutical composition comprising a nucleotide sequence according to claim 24, and a physiologically acceptable carrier.

30. The pharmaceutical composition according to claim 29, further comprising a nucleotide sequence comprising a sequence that is antisense to at least a portion of the fer gene.

31. The pharmaceutical composition according to claim 30, wherein the sequence that is antisense to at least a portion of the fer gene is SEQ ID NO 8.

32. The pharmaceutical composition according to claim 29, for use in the treatment of cancer.

33. The pharmaceutical composition according to claim 32, for use in the treatment of colorectal cancer.

34. The pharmaceutical composition according to claim 32, for use in the treatment of liver cancer.

35. A method for identifying a cancerous state in a cell comprising detecting FerC in the cell.

36. The method according to claim 35, wherein the cancer is colorectal cancer.

37. The method according to claim 35, comprising:
(a) extracting proteins from the cell;
(b) detecting Fer94 in the protein extract using a first antibody reactive with Fer94 and FerC; and
(c) detecting Fer94 in the protein extract using a second antibody reactive with Fer94 and not reactive with FerC;
the presence of a protein in the extract reactive to the first antibody and not reactive to the second antibody being indicative of the presence of FerC in the cell.

38. The method according to claim 37, wherein the second antibody binds to an epitope located at the N-terminus of Fer94.

39. A kit for identifying a cancerous state in a cell comprising:
(a) a first antibody reactive with Fer94 and FerC; and
(b) a second antibody reactive with Fer94 and not reactive with FerC.

40. A method of treating cancer comprising administering to an individual a pharmaceutical composition according to claim 29.

41. A polypeptide having the amino acid sequence SEQ ID NO 12.

42. A polypeptide having the amino acid sequence SEQ ID NO 14.

43. A nucleotide sequence encoding the polypeptide SEQ ID NO 12.

44. A nucleotide sequence encoding the polypeptide SEQ ID NO 14.

45. The nucleotide sequence according to claim 43, being SEQ ID NO. 11.

46. The nucleotide sequence according to claim 44, being SEQ ID NO. 13.


SEQUENCE LISTING (14 sequences)

SEQ ID NO: 1 - DNA, 30 bp, Homo sapiens
tcccttgcccagtaattctcccaatatgac

SEQ ID NO: 2 - DNA, 24 bp, Homo sapiens
aacccagtgccctcgaatcgatac

SEQ ID NO: 3 - DNA, 31 bp, Homo sapiens
ggacatattcaccaggtttcccatgactctc

SEQ ID NO: 4 - DNA, 31 bp, Homo sapiens
ccacatcagaagtccacagagatcaggaaag

SEQ ID NO: 5 - DNA, 31 bp, Homo sapiens
gcccgcgaattcacactcaaaagagaactac

SEQ ID NO: 6 - DNA, 22 bp, Homo sapiens
aaggtcatccctgagctgaacg

SEQ ID NO: 7 - DNA, 22 bp, Homo sapiens
caaaggtggaggagtgggtgtc

SEQ ID NO: 8 - RNA, 25 nt, Homo sapiens
acguauccaagucuuggcuacuuau

SEQ ID NO: 9 - RNA, 25 nt, Homo sapiens
cagcucugagccuuccacaucagaa

SEQ ID NO: 10 - RNA, 25 nt, Homo sapiens
gcccuaaguucagugaacuucagaa

SEQ ID NO: 11 - DNA, 1420 bp, Homo sapiens
ttggtaaattgacttccttgggcccactgtcactatctcttagctaaattttgagatggagcagaaaatgaaatgtcctcattgtaaggatcagctggagtctggtttcggctcccaatcctgtaaaacatgtgctttaatgttcagctctgagccttccacatcagaagtccacagagatcaggaaagaaaggagaggctatccaaatttgaatctattcgtcattcaattgctggaataattaggtctccaaaatctgcactgggctcttcagcactttctgatatgatctccatcagtgagaagcctttggcagaacaggactggtaccatggtgcaattcccagaatagaagctcaagaactgttaaaaaaacaaggagactttttggtgcgagagagtcatgggaaacctggtgaatatgtcctttctgtatattctgatggacagaggagacattttatcatacaatatgttgataacatgtatcgattcgagggcactgggttttcaaacattcctcaacttatagatcatcactatacaacaaaacaggtcatcactaagaaatcaggtgtagttctgctgaatcctattcctaaggacaagaaatggattctcagtcatgaagatgtcatattgggagaattactgggcaagggaaattttggtgaagtatataagggcacattaaaggataaaacttctgttgctgttaaaacatgtaaagaagatcttcctcaggaattgaaaataaaatttttacaagaagccaaaattctcaagcaatatgatcatcccaatattgtcaaacttataggagtttgcacacaaagacagcctgtctacatcattatggaactggtttcaggaggtgatttcctcacctttctgagaaggaagaaggatgaactaaaactcaaacagttagtgaaattttcattagacgctgctgctggtatgttgtatctcgagagtaaaaactgtatacacagggaccttgctgcaagaaactgcctggtaggtgaaaataatgttctgaaaatcagtgactttggaatgtctcgtcaagaggatggtggagtgtattcatcttctggcttaaagcagattcccattaaatggacagcaccggaagctcttaattatgggagatacagttcagagagtgacgtgtggagctttggcatccttctctgggagaccttcagcttaggggtttgtccgtaccctggaatgacaaatcagcaagcaagagagcaagtagaaagaggataccggatgtcagctccccagcactgtccagaggatatttccaaaatcatgatgaagtgttgggattataaacctgaaaatcgccctaagttcagtgaacttcagaaagagctcactatcatcaagagaaaactcacatagtga

SEQ ID NO: 12 - protein, 453 aa, Homo sapiens [sequence omitted]

SEQ ID NO: 13 - DNA, 129 bp, Homo sapiens
atggagcagaaaatgaaatgtcctcattgtaaggatcagctggagtctggtttcggctcccaatcctgtaaaacatgtgctttaatgttcagctctgagccttccacatcagaagtccacagagatcag

SEQ ID NO: 14 - protein, 43 aa, Homo sapiens
MEQKMKCPHCKDQLESGFGSQSCKTCALMFSSEPSTSEVHRDQ